SEQUENCE LISTING (12 sequences)

SEQ ID NO: 1 - DNA, 1978 bp, Homo sapiens
gcgccgggagaggcggcagcgcagccggacgcacagcgcagcgggccggcaccagctcggccgggcccggactcggactcggcggccggcgcggcgcggcccggcccgagcgagggtggggggcggcgggcggcgcggggcggcggcgagcgggggccatgcaggcgcgctactccgtgtccagccccaactccctgggagtggtgccctacctcggcggcgagcagagctactaccgcgcggcggccgcggcggccgggggcggctacaccgccatgccggcccccatgagcgtgtactcgcaccctgcgcacgccgagcagtacccgggcggcatggcccgcgcctacgggccctacacgccgcagccgcagcccaaggacatggtgaagccgccctatagctacatcgcgctcatcaccatggccatccagaacgccccggacaagaagatcaccctgaacggcatctaccagttcatcatggaccgcttccccttctaccgggacaacaagcagggctggcagaacagcatccgccacaacctctcgctcaacgagtgcttcgtcaaggtgccgcgcgacgacaagaagccgggcaagggcagctactggacgctggacccggactcctacaacatgttcgagaacggcagcttcctgcggcggcggcggcgcttcaagaagaaggacgcgttgaaggacaaggaggagaaggacaggctgcacctcaaggagccgcccccgcccggcgccagcccccgcccggcgccgccggagcaggccgacggcaacgcgcccggtccgcagccgccgcccgtgcgcatccaggacatcaagaccgagaacggtacgtgcccctcgccgccccagcccctgtccccggccgccgccttgggcagcggcagcgccgccgcggtgcccaagatcgagagccccgacagcagcagcagcagcctgtccagcgggagcagccccccgggcagcctgccgtcggcgcggccgctcagcctggacggtgcggattccgcgccgccgccgcccgcgccctccgccccgccgccgcaccatagccagggcttcagcgtggacaacatcatgacgtcgctgcgggggtcgccgcagagcgcggccgcggagctcagctccggccttctggcctcggcggccgcgtcctcgcgcgcggggatcgcacccccgctggcgctcggcgcctactcgcccggccagagctccctctacagctccccctgcagccagacctccagcgcgggcagctcgggcggcggcggcggcggcgcgggggccgcggggggcgcgggcggcgccgggacctaccactgcaacctgcaagccatgagcctgtacgcggccggcgagcgcgggggccacttgcagggcgcgcccgggggcgcgggcggctcggccgtggacgaccccctgcccgactactctctgcctccggtcaccagcagcagctcgtcgtccctgagtcacggcggcggcggcggcggcggcgggggaggccaggaggccggccaccaccctgcggcccaccaaggccgcctcacctcgtggtacctgaaccaggcgggcggagacctgggccacttggcgagcgcggcggcggcggcggcggccgcaggctacccgggccagcagcagaacttccactcggtgcgggagatgttcgagtcacagaggatcggcttgaacaactctccagtgaacgggaatagtagctgtcaaatggccttcccttccagccagtctctgtaccgcacgtccggagctttcgtctacgactgtagcaagttttgacacaccctcaaagccgaactaaatcgaaccccaaagcaggaaaagctaaaggaacccatcaaggcaaaatcgaaactaaaaaaaaaaaatccaattaaaaaaaacccctgagaatattcaccacaccagcgaacagaatatccctccaaaaattcagc

SEQ ID NO: 2 - protein, 553 aa, Homo sapiens [sequence omitted]

SEQ ID NO: 3 - DNA, 12 bp, Homo sapiens
agtaaataaaca

SEQ ID NO: 4 - DNA, 12 bp, Homo sapiens
agtaaacaaaca

SEQ ID NO: 5 - DNA, 20 bp, Homo sapiens
gcttcattccgaatcaccag

SEQ ID NO: 6 - DNA, 20 bp, Homo sapiens
gtcccctccctccaactatc

SEQ ID NO: 7 - DNA, 13 bp, Homo sapiens
gcccccgccgatg

SEQ ID NO: 8 - DNA, 13 bp, Homo sapiens
ggcgccgccgatg

SEQ ID NO: 9 - DNA, 12 bp, Homo sapiens
gggatatcgatg

SEQ ID NO: 10 - DNA, 12 bp, Homo sapiens
gggatatggatg

SEQ ID NO: 11 - DNA, 12 bp, Homo sapiens
agtagtggtacc

SEQ ID NO: 12 - DNA, 12 bp, Homo sapiens
agtactggtacc

(19) United States
(12) Patent Application Publication
(10) Pub. No.: US 2003/0013087 A1
(43) Pub. Date: Jan. 16, 2003

(54) NOVEL MUTATIONS IN THE FREAC3 GENE FOR DIAGNOSIS AND PROGNOSIS OF GLAUCOMA AND ANTERIOR SEGMENT DYSGENESIS

(76) Inventors: MICHAEL A. WALTER, EDMONTON (CA); TIM JORDAN, SOUTH HAMPTON (GB); VINCENT RAYMOND, QUEBEC CITY (CA)

Correspondence Address: KRISTINA BIEKER-BRADY PH D, CLARK & EBING LLP, 176 FEDERAL STREET, BOSTON, MA, 02110

(21) Appl. No.: 09/292,862

(22) Filed: Apr. 16, 1999

Related U.S. Application Data
(60) Provisional application No. 60/082,206, filed on Apr. 17, 1998.
(60) Provisional application No. 60/084,784, filed on May 8, 1998.

(51) Int. Cl. C12Q 1/68; A61K 31/00; G01N 33/567; A61K 31/70; G01N 33/53; A01N 43/04; C07H 21/04; A01N 61/00

(52) U.S. Cl. 435/6; 514/44; 536/23.5; 435/7.21; 435/975

(57) ABSTRACT

The invention features novel mutations in the FREAC3 gene. Our discovery provides methods for early diagnosis of glaucoma, other disorders of the eye, and heart defects. Also provided are cells having at least one deficient FREAC3 gene. Such cells may be used to detect therapeutic compounds that mimic FREAC3, are agonists of FREAC3, or otherwise modulate the level of FREAC3 biological activity.

### CROSS REFERENCE TO RELATED APPLICATIONS

This application claims priority from U.S. Provisional Application Serial No. 60/082,206, filed Apr. 17, 1998 and U.S. Provisional Application Serial No. 60/084,784, filed May 8, 1998.

### BACKGROUND OF THE INVENTION

Glaucoma, a major cause of blindness worldwide, is characterized by progressive degeneration of the optic nerve that is usually associated with increased intraocular pressure. In most cases, blindness from glaucoma begins with loss of peripheral vision. Central vision is maintained until the late disease stage. By the time visual loss is noted, damage is advanced.

Although glaucoma has a frequency of occurrence as high as those of high blood pressure and diabetes, the widespread lack of public awareness results in thousands of new cases of blindness annually. For example, the Canadian National Institute for the Blind has identified glaucoma as one of the two leading causes of blindness in Canada. And in the United States, over 1.2 million people have vision loss and over 80,000 people are legally blind as a result of glaucoma.

Fortunately, most cases of glaucoma can be successfully treated, and vision loss prevented, using existing drugs or surgical approaches. The key to successful treatment of glaucoma lies in its early detection, before irreversible optic nerve damage has occurred.

Anterior segment dysgenesis, the incorrect formation of the structures of the anterior segment of the eye, underlies the pathogenesis of some cases of congenital glaucoma. Glaucoma in patients with anterior segment dysgenesis is likely a result of incorrect regulation of the outflow of aqueous humor due to the improper development of the anterior segment angle structures.

Several autosomal dominant disorders of anterior segment formation that result in glaucoma recently have been genetically co-localized to chromosome 6p25. These disorders include iridogoniodysgenesis anomaly (IGDA), Axenfeld-Rieger Anomaly (ARA), familial glaucoma iridogoniodysplasia (FGI), and familial glaucoma with goniodysgenesis.

Given that glaucoma is a major cause of blindness worldwide, it would be desirable to have a simple diagnostic test to identify those at increased risk of blindness due to glaucoma. It would also be desirable to have experimental assays and animal models for the identification of compounds that are useful for the prevention or treatment of glaucoma.

### SUMMARY OF THE INVENTION

We report here the discovery of mutations in the FREAC3 genes of IRID1 patients. FREAC3 is a member of the forkhead/winged-helix transcription factor gene family. The results of expression studies and analyses of mice with homozygous knockouts of Mƒ1, the murine homologue of FREAC3, are consistent with a role for Mf1/FREAC3 in eye development and glaucoma.

Diagnosis of the genetic defect in IRID1 families, as provided herein, will allow immediate monitoring and pre-symptomatic treatment of the glaucoma that IRID1 patients often develop. Moreover, mutations of the IRID1 gene may be responsible for a significant portion of glaucoma patients not clinically diagnosed with IRID1, since not all IRID1 patients, even within IRID1 families, have the iris defects otherwise used to diagnosis IRID1.

Our discovery provides methods for early diagnosis of glaucoma and other disorders of the eye. Also provided are cells having at least one deficient FREAC3 gene. Such cells may be used to detect new therapeutic compounds that mimic FREAC3, are agonists of FREAC3, or otherwise increase the level of FREAC3 biological activity.

In a first aspect, the invention features a method of diagnosing a mammal for an increased likelihood of developing a disease of the eye, comprising analyzing nucleic acid of the mammal to determine whether the nucleic acid contains a mutation in a FREAC3 gene. The presence of a mutation is an indication that the mammal has an increased likelihood of developing a disease of the eye.

In a second aspect, the invention features a method of diagnosing a mammal for an increased likelihood of having a developmental defect, comprising analyzing nucleic acid of the mammal to determine whether the nucleic acid contains a mutation in a FREAC3 gene. The presence of a mutation is an indication that the mammal has an increased likelihood of having a developmental defect.

In preferred embodiments of the second aspect of the invention, the developmental defect is a cardiac defect or is anterior segment dysgenesis.

In a preferred embodiment of the first and second aspects of the invention, the mutation is a missense mutation. For example, the missense mutation may be a G to C transversion at coding nucleotide 245, which results in a Ser82Thr mutation in helix 1 of the FREAC3 forkhead domain, or the missense mutation may be a G to C transversion at coding nucleotide 261, which results in a I1e87Met mutation in helix 1 of the FREAC3 forkhead domain. In another embodiment of the first and second aspects, the mutation may be a frameshift mutation. For example, the frameshift mutation may result from a ten-base-pair deletion of coding nucleotides 93 through 102. A frameshift mutation may result in a truncated protein.

In other embodiments of the first and second aspects, primers may be used for detecting the mutation, such primers may be selected from those shown in Table 1.

The methods of the first and second aspects may further comprise the step of sequencing nucleic acid encoding FREAC3 from the mammal. In addition, the methods may further comprise the step of using nucleic acid primers specific for the FREAC3 gene, which are used for DNA amplification by the polymerase chain reaction.

In still further embodiments of the first and second aspects, the analyzing includes detecting the loss of a recognition site for a restriction endonuclease (e.g., Alu I), or the analyzing includes detecting the gain of a recognition site for a restriction endonuclease (e.g., Bsp HI). The analyzing may also include detecting a loss of one or more nucleotides, or a gain of one or more nucleotides. Furthermore, the analyzing may include mismatch detection, using single strand conformational polymorphism (SSCP) analysis, or restriction fragment length polymorphism (RFLP) analysis.

In a third, related aspect, the invention features a kit for the analysis of FREAC3 nucleic acid. The kit comprises nucleic acid probes for analyzing the nucleic acid of a mammal, wherein the analyzing is sufficient to determine whether the mammal contains a mutation in FREAC3 nucleic acid.

In a fourth aspect, the invention features a method of making an antibody that specifically binds a mutant FREAC3 polypeptide, comprising administering a mutant FREAC3 polypeptide, or fragment thereof, to an animal capable of generating an immune response, and isolating the antibody from the animal.

In a fifth aspect, the invention features a method of detecting the presence of a mutant FREAC3 polypeptide, comprising contacting a sample with an antibody that specifically binds a mutant FREAC3 polypeptide and assaying for binding of the antibody to the mutant polypeptide.

In preferred embodiments of the fifth aspect, the mutant FREAC3 polypeptide may have a threonine residue at FREAC3 amino acid position 82, or a methionine residue at FREAC3 amino acid position 87, or the mutant FREAC3 polypeptide may have an amino acid sequence that differs from the FREAC3 wild-type sequence, wherein the amino acid sequence that differs is carboxy-terminal to FREAC3 amino acid 33 (ala 33).

In a sixth aspect, the invention features a method of diagnosing a mammal for an increased likelihood of developing a disease of the eye, comprising detecting the presence of a mutant FREAC3 polypeptide in the mammal. The presence of a mutant FREAC3 polypeptide indicates that the mammal has a mutation in a FREAC3 gene, and the presence of a mutation is an indication that the mammal has an increased likelihood of developing a disease of the eye.

In a seventh aspect, the invention features a method of diagnosing a mammal for an increased likelihood of having a developmental defect, comprising detecting the presence of a mutant FREAC3 polypeptide in the mammal. The presence of a mutant FREAC3 polypeptide indicates that the mammal has a mutation in a FREAC3 gene, and the presence of a mutation is an indication that the mammal has an increased likelihood of having a developmental defect.

In an eighth aspect, the invention features a kit for the analysis of FREAC3 nucleic acid, comprising antibodies for analyzing the polypeptides of a mammal, wherein the analyzing is sufficient to determine whether the mammal contains a mutation in FREAC3 nucleic acid.

In a ninth aspect, the invention features nucleic acid encoding mutant FREAC3. The nucleic acid has at least one mutation, and the mutation is an indication that a mammal from which the nucleic acid is derived has an increased likelihood of developing glaucoma.

In various embodiments of the ninth aspect of the invention, the mutation may be a G to C transversion at coding nucleotide 245, or a G to C transversion at coding nucleotide 261, or a deletion of coding nucleotides 93 through 102. In another embodiment, the nucleic acid is operably linked to regulatory sequences for expression of FREAC3, and the regulatory sequences comprise a promoter.

In preferred embodiments of the first, second, third, sixth, seventh, eight, and ninth aspects of the invention, the mammal is human, or the mammal is prenatal.

In a tenth aspect, the invention features a cell containing the nucleic acid of the ninth aspect of the invention.

In further embodiments of the tenth aspect of the invention, the cell may be a prokaryotic cell, or a eukaryotic cell, such as a yeast cell or a mammalian cell. In another embodiment of the tenth aspect, the promoter may be inducible.

In an eleventh aspect, the invention features a non-human transgenic mammal containing the nucleic acid of the ninth aspect of the invention. The nucleic acid is operably linked to regulatory sequences for expression of FREAC3.

In a preferred embodiment of the eleventh aspect, the mammal may be a rodent. In another preferred embodiment, one or both endogenous alleles encoding a FREAC3 polypeptide may be disrupted, deleted, or otherwise rendered nonfunctional.

In a related, twelfth aspect, the invention features cells from the transgenic mammal of the eleventh aspect of the invention.

In a thirteenth aspect, the invention features a non-human mammal in which one or both endogenous alleles encoding a FREAC3 polypeptide are mutated at positions corresponding to those shown in FIG. 2.

In a related, fourteenth aspect, the invention features cells from the mammal of the thirteenth aspect of the invention.

In a fifteenth aspect, the invention features a method of detecting a compound useful for the prevention or treatment of a disease of the eye, comprising assaying transcription levels of a reporter gene operably linked to a promoter, wherein the promoter contains a FREAC3 binding site. The method comprises the steps of: (a) exposing the reporter gene to the compound, and (b) assaying the reporter gene for an alteration in reporter gene activity relative to a reporter gene not exposed to the compound.

In various embodiments of the fifteenth aspect of the invention, the reporter gene may be in a cell, the cell may be in an animal, and an increase in transcription indicates a compound useful for the prevention or treatment of glaucoma.

In a preferred embodiment of the first, sixth, and fifteenth aspects of the invention, the disease of the eye is glaucoma.

In a sixteenth aspect, the invention features a method of treating a disease of the eye by in vivo gene therapy, comprising introducing into the cells of the eye a nucleic acid that encodes wild-type FREAC3. The nucleic acid is operably linked to regulatory sequences for expression of FREAC3, the regulatory sequences comprise a promoter, and expression of FREAC3 is sufficient to ameliorate symptoms of the disease.

In preferred embodiments of the sixteenth aspect, the nucleic acid may be introduced in to the cells by means of a viral vector, that contains the nucleic acid encoding FREAC3, or the nucleic acid may be introduced into the cells by transformation.

By “FREAC3 nucleic acid” or “FREAC3 gene” is meant a nucleic acid, such as genomic DNA, cDNA, or mRNA, that encodes FREAC3, a FREAC3 protein, FREAC3 polypeptide, or portion thereof, as defined below. A FREAC3 nucleic acid also may be a FREAC3 primer or probe, or antisense nucleic acid that is complementary to an mRNA encoding FREAC3.

By “wild-type FREAC3” is meant a FREAC3 nucleic acid or FREAC3 polypeptide having the nucleic acid and/or amino acid sequence most often observed among members of a given animal species and not associated with a disease phenotype. Wild-type FREAC3 is biologically active FREAC3. A wild-type FREAC3 is, for example, a FREAC3 polypeptide or nucleic acid that has the sequence shown in FIG. 2. Wild-type FREAC3 also may be polymorphic FREAC3 as described herein (i.e., insertion of an extra GGC (glycine) codon after coding nucleotide 345 or 447, as described below).

By “mutant FREAC3” “FREAC3 mutation(s)” or “mutations in FREAC3” is meant a FREAC3 polypeptide or nucleic acid having a sequence that deviates from the wild-type sequence in a manner sufficient to confer an increased likelihood of developing anterior segment malformations and/or glaucoma in at least some genetic and/or environmental backgrounds. Such mutations may be naturally occurring, or artificially induced. They may be, without limitation, insertion, deletion, frameshift, or missense mutations; such mutations may result in replacement of a wild-type amino acid with a different amino acid, or premature termination of the polypeptide. A mutant FREAC3 protein may have one or more mutations, and such mutations may affect different aspects of FREAC3 biological activity (protein function), to various degrees. Alternatively, a FREAC3 mutation may indirectly affect FREAC3 biological activity by influencing, for example, the transcriptional activity of a gene encoding FREAC3, or the stability of FREAC3 mRNA. For example, a mutant FREAC3 gene may be a gene which expresses a mutant FREAC3 protein or may be a gene which alters the level of FREAC3 protein in a manner sufficient to confer a statistically significant (p≦0.05) increased likelihood of developing glaucoma in at least some genetic and/or environmental backgrounds.

By “biologically active FREAC3” is meant that the FREAC3 within an individual is sufficient to prevent anterior segment dysgenesis or development/progression of FREAC3-dependent glaucoma in an otherwise-healthy individual. An assessment of the relative FREAC3 biological activity in an individual may be made, e.g., by comparing the FREAC3 sequence in the individual to known wild type and mutant FREAC3 sequences, by measuring the relative amount of FREAC3 binding in a sample to a FREAC3 binding site (e.g., aGTAAA(T/c)AAAca), or by measuring the relative ability of FREAC3 in a sample to transactivate expression of a FREAC3-dependent gene (e.g., by measuring reporter gene expression from a chimeric gene that contains a FREAC3 binding site in its regulatory region), relative to that of wild-type FREAC3, or by equivalent approaches. Prevention and/or treatment of glaucoma may be effected by increasing the biological activity of a FREAC3 molecule or by increasing the number of FREAC3 molecules in a patient with relatively low FREAC3 biological activity. Preferably, FREAC3 biological activity is at least 25% of that in a normal individual, more preferably, at least 50%, even more preferably, at least 75%, and most preferably, at least 90% of that in a normal individual.

By “Mf1” is meant the mouse homolog of human FREAC3. The definitions set forth above for FREAC3 (e.g., “wild-type”, “mutated”) apply to Mf1.

By “endogenous allele” is meant a copy of a gene that has not been inserted into a genome by artifice.

By “missense mutation” is meant the substitution of one purine or pyrimidine base (i.e. A, T, G, or C) by another within a nucleic acid sequence, such that the resulting new codon encodes an amino acid distinct from the amino acid originally encoded by the reference (e.g. wild-type) codon.

By “frameshift mutation” is meant the insertion or deletion of at least one nucleotide within a polynucleotide coding sequence. A frameshift mutation alters the codon reading frame at and/or downstream from the mutation site. Such a mutation results either in the substitution of the encoded wild-type amino acid sequence by a novel amino acid sequence, or a premature termination of the encoded polypeptide due to the creation of a stop codon, or both.

By “high stringency conditions” is meant conditions that allow hybridization comparable with the hybridization that occurs during an overnight incubation using a DNA probe of at least 500 nucleotides in length, in a solution containing 0.5 M NaHPO₄, pH 7.2, 7% SDS, 1 mM EDTA, 1% BSA (fraction V), and 100 μg/ml denatured, sheared salmon sperm DNA, at a temperature of 65° C., or a solution containing 48% formamide, 4.8× SSC (150 mM NaCl, 15 mM trisodium citrate), 0.2 M Tris-Cl, pH 7.6, 1× Denhardt's solution, 10% dextran sulfate, 0.1% SDS, and 100 μg/ml denatured, sheared salmon sperm DNA, at a temperature of 42° C. (these are typical conditions for high stringency Northern or Southern, or colony hybridizations). High stringency hybridization may be used for techniques such as high stringency PCR, DNA sequencing, single strand conformational polymorphism analysis, and in situ hybridization. The immediately aforementioned techniques are usually performed with relatively short probes (e.g., usually 16 nucleotides or longer for PCR or sequencing, and 40 nucleotides or longer for in situ hybridization). The high stringency conditions used in these techniques are well known to those skilled in the art of molecular biology, and may be found, for example, in F. Ausubel et al., Current Protocols in Molecular Biology, John Wiley & Sons, New York, N.Y., 1997, hereby incorporated by reference.

By “analyzing” or “analysis” is meant subjecting a FREAC3 nucleic acid or FREAC3 polypeptide to a test procedure that allows the determination of whether a FREAC3 gene is wild-type or mutant. For example, one could analyze the FREAC3 genes of an animal by amplifying genomic DNA using the polymerase chain reaction, and then determining the DNA sequence of the amplified DNA.

By “assaying” is meant analyzing the effect of a treatment or exposure, be it chemical or physical, administered to whole animals or cells derived therefrom. The material being analyzed may be an animal, a cell, a lysate or extract derived from a cell, or a molecule derived from a cell. The analysis may be, for example, for the purpose of detecting altered gene expression, altered nucleic acid stability (e.g. mRNA stability), altered protein stability, altered protein levels, or altered protein biological activity. The means for analyzing may include, for example, nucleic acid amplification techniques, reporter gene assays, antibody labeling, immunoprecipitation, and phosphorylation assays and other techniques known in the art for conducting the analysis of the invention.

By “modulating” is meant changing, either by decrease or increase.

By “probe” or “primer” is meant a single-stranded DNA or RNA molecule of defined sequence that can base-pair to a second DNA or RNA molecule that contains a complementary sequence (the “target”). The stability of the resulting hybrid depends upon the extent of the base pairing that occurs. The extent of base-pairing is affected by parameters such as the degree of complementarity between the probe and target molecules, and the degree of stringency of the hybridization conditions. The degree of hybridization stringency is affected by parameters such as temperature, salt concentration, and the concentration of organic molecules such as formamide, and is determined by methods known to one skilled in the art Probes or primers specific for FREAC3 nucleic acid preferably will have at least 35% sequence identity, more preferably at least 45-55% sequence identity, still more preferably at least 60-75% sequence identity, still more preferably at least 80-90% sequence identity, and most preferably 100% sequence identity. Probes may be detectably-labelled, either radioactively, or non-radioactively, by methods well-known to those skilled in the art. Probes are used for methods involving nucleic acid hybridization, such as: nucleic acid sequencing, nucleic acid amplification by the polymerase chain reaction, single stranded conformational polymorphism (SSCP) analysis, restriction fragment polymorphism (RFLP) analysis, Southern hybridization, Northern hybridization, in situ hybridization, electrophoretic mobility shift assay (EMSA).

By “mismatch detection approach” is meant identification of a mutation (i.e., mismatch) in a gene using standard techniques to analyze nucleic acid from a patient sample. Generally, these techniques involve PCR amplification of nucleic acid from the patient sample, followed by identification of the mutation by either altered hybridization, aberrant electrophoretic gel migration, binding or cleavage mediated by mismatch binding proteins, direct nucleic acid sequencing, or other techniques that are known in the art.

By “pharmaceutically acceptable carrier” means a carrier which is physiologically acceptable to the treated mammal while retaining the therapeutic properties of the compound with which it is administered. One exemplary pharmaceutically acceptable carrier is physiological saline. Other physiologically acceptable carriers and their formulations are known to one skilled in the art and described, for example, in Remington's Pharmaceutical Sciences, (18^{th} edition), ed. A. Gennaro, 1990, Mack Publishing Company, Easton, Pa.

By “identity” is meant that a polypeptide or nucleic acid sequence possesses the same amino acid or nucleotide residue at a given position, compared to a reference polypeptide or nucleic acid sequence to which the first sequence is aligned. Sequence identity is typically measured using sequence analysis software with the default parameters specified therein, such as the introduction of gaps to achieve an optimal alignment (e.g., Sequence Analysis Software Package of the Genetics Computer Group, University of Wisconsin Biotechnology Center, 1710 University Avenue, Madison, Wis. 53705).

Sequence identity is typically measured using sequence analysis software with the default parameters specified therein (e.g., Sequence Analysis Software Package of the Genetics Computer Group, University of Wisconsin Biotechnology Center, 1710 University Avenue, Madison, Wis. 53705). This software program matches similar sequences by assigning degrees of homology to various substitutions, deletions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

By “substantially identical” is meant a polypeptide or nucleic acid exhibiting at least 50%, preferably 85%, more preferably 90%, and most preferably 95% identity to a reference amino acid or nucleic acid sequence. For polypeptides, the length of comparison sequences will generally be at least 16 amino acids, preferably at least 20 amino acids, more preferably at least 25 amino acids, and most preferably 35 amino acids. For nucleic acids, the length of comparison sequences will generally be at least 50 nucleotides, preferably at least 60 nucleotides, more preferably at least 75 nucleotides, and most preferably 110 nucleotides.

By “substantially pure polypeptide” is meant a polypeptide that has been separated from the components that naturally accompany it. Typically, the polypeptide is substantially pure when it is at least 60%, by weight, free from the proteins and naturally-occurring organic molecules with which it is naturally associated. Preferably, the polypeptide is a FREAC3 polypeptide that is at least 75%, more preferably at least 90%, and most preferably at least 99%, by weight, pure. A substantially pure FREAC3 polypeptide may be obtained, for example, by extraction from a natural source (e.g., a peripheral blood leukocyte), by expression of a recombinant nucleic acid encoding a FREAC3 polypeptide, or by chemically synthesizing the protein. Purity can be measured by any appropriate method, e.g., by column chromatography, polyacrylamide gel electrophoresis, or HPLC analysis.

A protein is substantially free of naturally associated components when it is separated from those contaminants which accompany it in its natural state. Thus, a protein which is chemically synthesized or produced in a cellular system different from the cell from which it naturally originates will be substantially free from its naturally associated components. Accordingly, substantially pure polypeptides not only includes those derived from eukaryotic organisms but also those synthesized in E. coli or other prokaryotes.

By “substantially pure DNA” is meant DNA that is free of the genes which, in the naturally-occurring genome of the organism from which the DNA of the invention is derived, flank the gene. The term therefore includes, for example, a recombinant DNA which is incorporated into a vector; into an autonomously replicating plasmid or virus; or into the genomic DNA of a prokaryote or eukaryote; or which exists as a separate molecule (e.g., a cDNA or a genomic or cDNA fragment produced by PCR or restriction endonuclease digestion) independent of other sequences. It also includes a recombinant DNA which is part of a hybrid gene encoding additional polypeptide sequence.

By “transgene” is meant any piece of DNA which is inserted by artifice into a cell, and becomes part of the genome of the organism which develops from that cell. Such a transgene may include a gene which is partly or entirely heterologous (i.e., foreign) to the transgenic organism, or may represent a gene homologous to an endogenous gene of the organism.

By “transgenic” is meant any cell which includes a DNA sequence which is inserted by artifice into a cell and becomes part of the genome of the organism which develops from that cell. As used herein, the transgenic organisms are generally transgenic mammals (e.g., rodents such as rats or mice) and the DNA (transgene) is inserted by artifice into the nuclear genome.

By “knockout mutation” is meant an alteration in the nucleic acid sequence that reduces the biological activity of the polypeptide normally encoded therefrom by at least 80% relative to the unmutated gene. The mutation may, without limitation, be an insertion, deletion, frameshift mutation, or a missense mutation. Preferably, the mutation is an insertion or deletion, or is a frameshift mutation that creates a stop codon.

By “transformation” is meant any method for introducing foreign molecules into a cell. Lipofection, DEAE-dextran-mediated transfection, microinjection, protoplast fusion, calcium phosphate precipitation, retroviral delivery, electroporation, and biolistic transformation are just a few of the methods known to those skilled in the art which may be used. For example, biolistic transformation is a method for introducing foreign molecules into a cell using velocity driven microprojectiles such as tungsten or gold particles. Such velocity-driven methods originate from pressure bursts which include, but are not limited to, helium-driven, air-driven, and gunpowder-driven techniques. Biolistic transformation may be applied to the transformation or transfection of a wide variety of cell types and intact tissues including, without limitation, intracellular organelles (e.g., and mitochondria and chloroplasts), bacteria, yeast, fungi, algae, animal tissue, and cultured cells.

By “transformed cell” is meant a cell into which (or into an ancestor of which) has been introduced, by means of recombinant DNA techniques, a DNA molecule encoding (as used herein) a FREAC3 polypeptide.

By “positioned for expression” is meant that the DNA molecule is positioned adjacent to a DNA sequence which directs transcription and translation of the sequence (i.e., facilitates the production of, e.g., a FREAC3 polypeptide, a recombinant protein or a RNA molecule).

By “promoter” is meant a minimal sequence sufficient to direct transcription. Also included in the invention are those promoter elements which are sufficient to render promoter-dependent gene expression controllable for cell type-specific, tissue-specific, temporal-specific, or inducible by external signals or agents; such elements may be located in the 5′ or 3′ or intron sequence regions of the native gene.

By “operably linked” is meant that a gene and one or more regulatory sequences are connected in such a way as to permit gene expression when the appropriate molecules (e.g., transcriptional activator proteins) are bound to the regulatory sequences.

By “FREAC3 binding site” is meant binding a DNA sequence that allows specific binding of FREAC3 to the DNA sequence. One FREAC3 binding site is the sequence aGTAAA(T/c)AAAca (SEQ ID NOs: 3 and 4).

By “detectably-labeled” is meant any means for marking and identifying the presence of a molecule, e.g., an oligonucleotide probe or primer, a gene or fragment thereof, or a cDNA molecule. Methods for detectably-labeling a molecule are well known in the art and include, without limitation, radioactive labeling (e.g., with an isotope such as ³²P, ³³P or ³⁵S) and nonradioactive labeling (e.g., chemiluminescent labeling, e.g., fluorescein labeling).

By “purified antibody” is meant antibody which is at least 60%, by weight, free from proteins and naturally occurring organic molecules with which it is naturally associated. Preferably, the preparation is at least 75%, more preferably 90%, and most preferably at least 99%, by weight, antibody, e.g., a mutant FREAC3-specific antibody. A purified antibody may be obtained, for example, by affinity chromatography using recombinantly-produced protein or conserved motif peptides and standard techniques.

By “specifically binds” is meant an antibody that recognizes and binds a FREAC3 polypeptide but does not bind unrelated polypeptides. A preferred antibody specifically binds a mutant or polymorphic FREAC3 polypeptide but does not substantially recognize and bind wild-type FREAC3 molecules in a sample, e.g., a biological sample, that naturally includes protein. A preferred antibody binds to a FREAC3 polypeptide having a mutation or polymorphism at one or more of the positions indicated in FIG. 2.

By “coding nucleotide” is meant a nucleotide within the coding region of FREAC3. For example, the first residue of the initiator methionine codon of FREAC3 is coding nucleotide 1, and the first residue of the second codon in FREAC3 is coding nucleotide 4. The numbering of all coding nucleotides is relative to coding nucleotide 1.

### BRIEF DESCRIPTION OF THE DRAWINGS

FIG. 1 is a diagram showing the genetic mapping of the IRID1 gene(s).

FIG. 2 is a diagram showing the cDNA and amino acid sequence of FREAC3.

FIG. 3 is a diagram showing autoradiographs that display the sequences of mutated FREAC3 genes.

FIG. 4 is a diagram showing Northern blot analyses of FREAC3 expression in human tissues.

FIGS. 5(a-d) is a diagram showing expression studies of the FREAC3 mouse homologue Mf1 in the developing murine eye.

### DESCRIPTION OF THE PREFERRED EMBODIMENTS

Genetic linkage, genome mismatch scanning, and analysis of patients with chromosomal alterations of chromosome 6 have indicated that a major locus for development of the anterior segment of the eye and for development of glaucoma, IRID1, is located at 6p25. FREAC3, a member of the forkhead/winged helix transcription factor family, has also been mapped to 6p25. DNA sequencing of FREAC3 in five IRID1 families and 16 sporadic patients with anterior segment defects revealed three mutations: a 10 base pair deletion predicted to cause a frameshift and premature protein truncation prior to the FREAC3 forkhead DNA-binding domain and two missense mutations of conserved amino acids within the FREAC3 forkhead domain. These missense mutations could impair DNA binding and nuclear localization of the FREAC3 protein. Our finding of FREAC3 mutations in three patients with ocular defects indicates that FREAC3 is involved in anterior segment dysgenesis and glaucoma. Although numerous human forkhead family genes have been described, ours is the first showing that a mutation in a forkhead gene underlies a human developmental disorder.

Mf1, the murine homologue of FREAC3, is expressed in the developing brain, skeletal system and eye, consistent with FREAC3 having a role in ocular development. The three presumed inactivating mutations of FREAC3 and the expression pattern of Mf1 in the developing eye are consistent with haplo-insufficiency of FREAC3 underlying the autosomal dominant glaucoma and anterior segment dysgenesis in IRID1 patients.

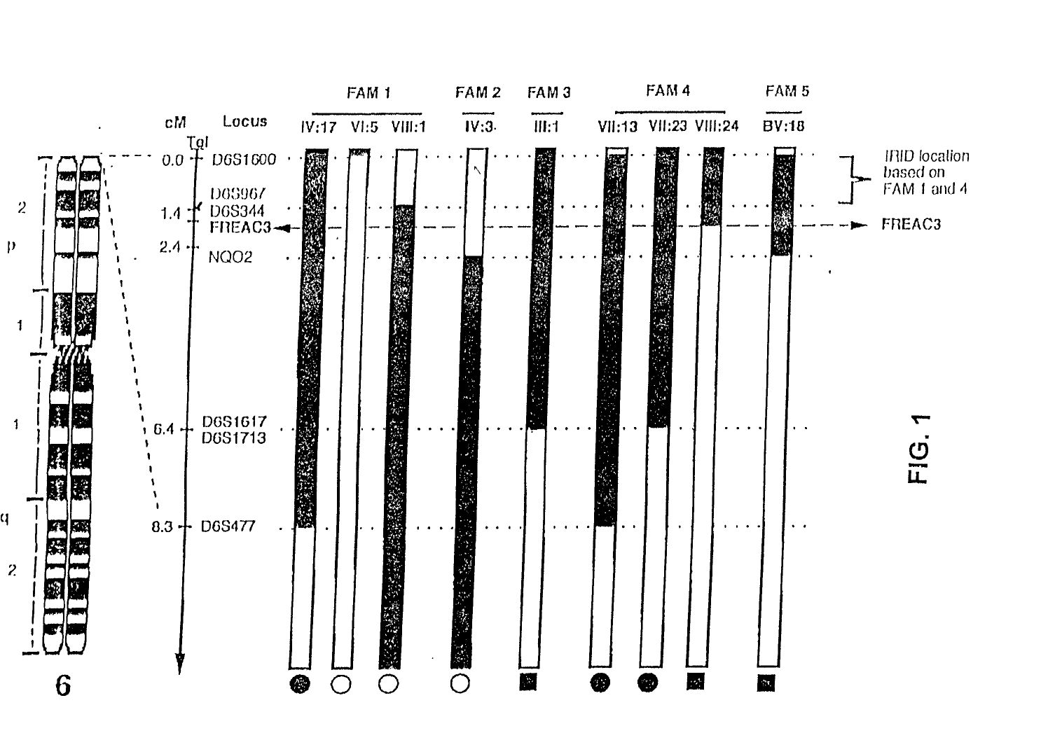

All three mutations found in the FREAC3 gene occurred in patients (a family, and two sporadic cases) originally diagnosed with the Axenfeld-Rieger anomaly (ARA) form of IRID1. Mutational screening, however, excluded FREAC3 from underlying the anterior segment disorders in four other families with glaucoma and anterior segment dysgenesis linked to 6p25, and genetic linkage analyses actually excluded the FREAC3 gene in two of these families (FIG. 1). Interestingly these four families (IRID1 families 1, 2, 4 and 5) were originally diagnosed with IGDA, IGDA, FGI, and familial glaucoma with goniodysgenesis, respectively.

The FREAC3 mutations described above were found in patients with ARA. While all IRID1 autosomal dominant disorders include glaucoma, iris hypoplasia and anterior angle defects, ARA patients additionally present with a prominent, anteriorly displaced Schwalbe's line attached to peripheral iris strands bridging the iridocorneal angle, and displaced pupils, features not typically seen in IGDA, FGI, or familial goniodsygenesis. The four remaining IRID1 families might thus be phenotypically as well as genetically distinct from the ARA family and patients found to carry FREAC3 mutations. Our findings demonstrate that while mutations of FREAC3 result in anterior segment defects and glaucoma in some patients, at least one more locus involved in the regulation of eye development must also be located at 6p25.

Knowledge of the gene defect in IRID1 families will allow immediate monitoring and pre-symptomatic treatment of the glaucoma that IRID1 patients often develop. Moreover, mutations of the IRID1 gene may be responsible for a significant portion of glaucoma patients not clinically diagnosed with IRID1, since not all IRID1 patients, even within IRID1 families, have the iris defects used to diagnose IRID1. As the glaucoma that IRID1 patients develop is often difficult to treat with existing drugs, detection of a FREAC3 mutation in a patient could indicate that the patient's glaucoma should be treated surgically. In the future, detection of FREAC3 mutations in glaucoma patients could permit the separation of patients into different glaucoma sub-groups, not only allowing improved prediction of patient response to different glaucoma treatments, but also the design of better glaucoma treatments. Thus the characterization of the FREAC3 gene will not only greatly increase our understanding of the development of the anterior segment, but also that of the pathogenesis of glaucoma.

Detection of FREAC3 Mutations and Altered Expression Levels

FREAC3 polypeptides and nucleic acid sequences are of diagnostic use in identifying patients that have an increased likelihood of having anterior segment dysgenesis and/or developing glaucoma. Mutations in FREAC3 that decrease FREAC3 expression or biological activity may be correlated with anterior segment defects and glaucoma in humans.

A biological sample obtained from a patient may be analyzed for one or more mutations in FREAC3 nucleic acid sequences using a mismatch detection approach (such mutations may also be detected in prenatal screens). Generally, these techniques involve PCR amplification of FREAC3 genomic DNA or RT-PCR amplification of FREAC3 mRNA from the patient sample, followed by identification of the mutation (i.e., mismatch) by either altered hybridization, aberrant electrophoretic gel migration, binding or cleavage mediated by mismatch binding proteins, or direct nucleic acid sequencing. Any of these techniques may be used to facilitate mutant FREAC3 detection, and each is well known in the art; examples of particular techniques are described, without limitation, in Orita et al. (Proc. Natl. Acad. Sci. USA 86:2766-2770, 1989) and Sheffield et al. (Proc. Natl. Acad. Sci. USA 86:232-236, 1989).

Mismatch detection assays provide an opportunity to diagnose a FREAC3-mediated predisposition to glaucoma before the onset of symptoms. For example, a patient heterozygous for a FREAC3 mutation that decreases FREAC3 biological activity or expression may show no clinical symptoms and yet possess a higher than normal probability of developing glaucoma. Moreover, certain wild-type alleles of FREAC3 present in the population may enhance the risk of developing other eye diseases. Given this diagnosis, a patient may take precautions to minimize their exposure to adverse environmental factors (for example, UV exposure), to carefully monitor their medical condition (for example, through frequent physical examinations) and to take additional preventative measures, such as using prophylactic medication or undergoing surgery or other preventative treatment.

A decrease in the level of FREAC3 production also may provide an indication of a deleterious or potentially deleterious condition in a patient. Levels of FREAC3 expression may be assayed by any standard technique. For example, FREAC3 transcriptional regulatory sequences may be analyzed for mutations that alter expression levels as a means of determining whether altered expression is likely, or FREAC3 transcription may be quantified in normal cells (e.g. peripheral blood leukocytes) and these levels may be compared to FREAC3 transcription levels in the peripheral blood leukocytes of the test subject. FREAC3 expression in a biological sample (e.g., a biopsy) may be monitored by standard Northern blot analysis or by PCR (see, e.g., F. Ausubel et al., Current Protocols in Molecular Biology, John Wiley & Sons, New York, N.Y., 1998; PCR Technology: Principles and Applications for DNA Amplification, H. A. Ehrlich, Ed., Stockton Press, NY; Yap et al. Nucl. Acids. Res. 19:4294, 1991).

The FREAC3 expression assays described above may be carried out using any biological sample (for example, any biopsy sample, blood sample, or other cell or tissue sample) in which FREAC3 is normally expressed. Identification of a mutated FREAC3 gene may also be assayed using these sources for test samples. Low levels of FREAC3 expression, or a mutation in a FREAC3 gene identifies a patient at increased risk for anterior segment dysgenesis and glaucoma.

Alternatively, a FREAC3 mutation, particularly as part of a diagnosis for predisposition to FREAC3-associated degenerative disease, may be tested using a DNA sample from any cell, for example, by mismatch detection techniques. Preferably, the DNA sample is subjected to PCR amplification prior to analysis.

In yet another approach, immunoassays are used to detect or monitor FREAC3 protein expression in a biological sample. FREAC3-specific polyclonal or monoclonal antibodies (produced by standard techniques) may be used in any standard immunoassay format (e.g., ELISA, Western blot, or RIA) to measure FREAC3 polypeptide levels. These levels would be compared to wild-type FREAC3 levels. For example, a decrease in FREAC3 production may indicate an increased risk of developing glaucoma. Examples of immunoassays are described, e.g., in F. Ausubel et al., Current Protocols in Molecular Biology, John Wiley & Sons, New York, N.Y., 1998.

Immunohistochemical techniques may also be utilized for FREAC3 detection. For example, a tissue sample may be obtained from a patient, sectioned, and stained for the presence of FREAC3 using an anti-FREAC3 antibody and any standard detection system (e.g., one which includes a secondary antibody conjugated to horseradish peroxidase). General guidance regarding such techniques can be found in, e.g., Bancroft and Stevens (Theory and Practice of Histological Techniques, Churchill Livingstone, 1982) and F. Ausubel et al., Current Protocols in Molecular Biology, John Wiley & Sons, New York, N.Y., 1994.

Assays for the Identification of Compounds that Modulate or Mimic FREAC3 Biological Activity

Methods of observing changes in FREAC3 biological activity are exploited in high-throughput assays for the purpose of identifying compounds that modulate mutant or wild-type FREAC3 transcriptional activity. Compounds that mimic FREAC3 activity also may be identified by such assays. Furthermore, compounds that modulate transcription of the FREAC3 gene itself may be identified; in some cases, it may be desirable to increase or decrease FREAC3 protein levels (e.g., decrease mutant FREAC3 levels or increase wild-type levels). Such identified compounds may have utility as therapeutic agents in the treatment or prevention of glaucoma or anterior segment dysgenesis.

Test Compounds

In general, novel drugs for prevention or treatment of anterior segment dysgenesis or glaucoma that work by modulating or mimicking FREAC3 biological activity are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their therapeutic value in treating or preventing glaucoma or anterior segment dysgenesis should be employed whenever possible.

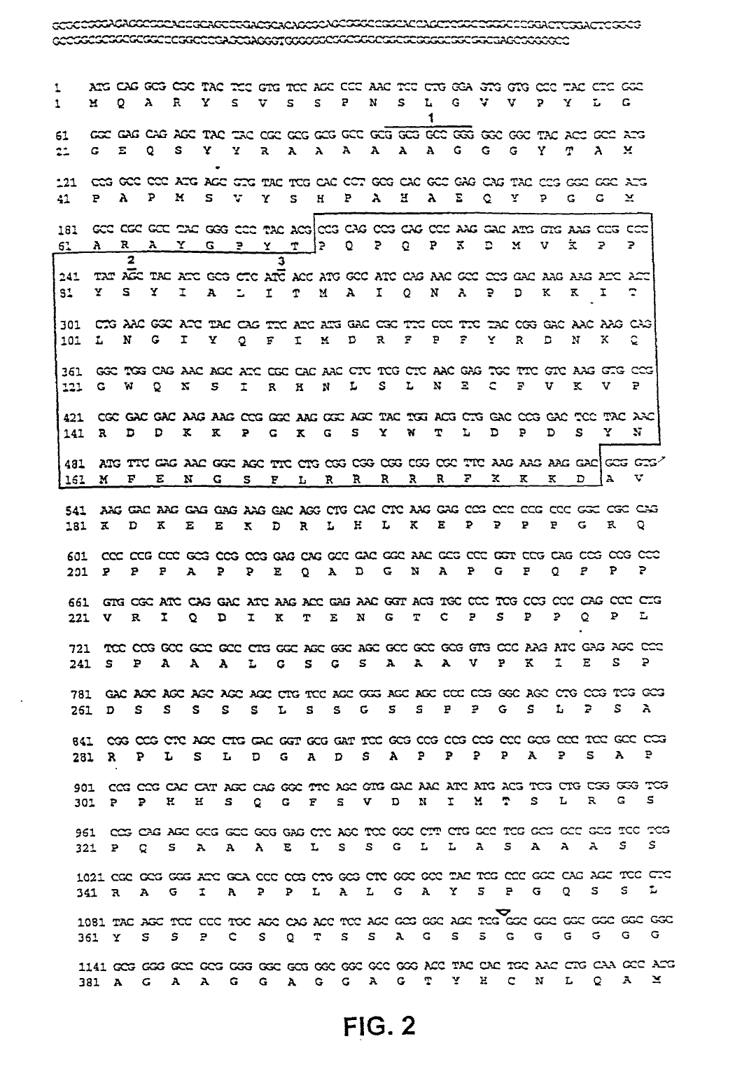

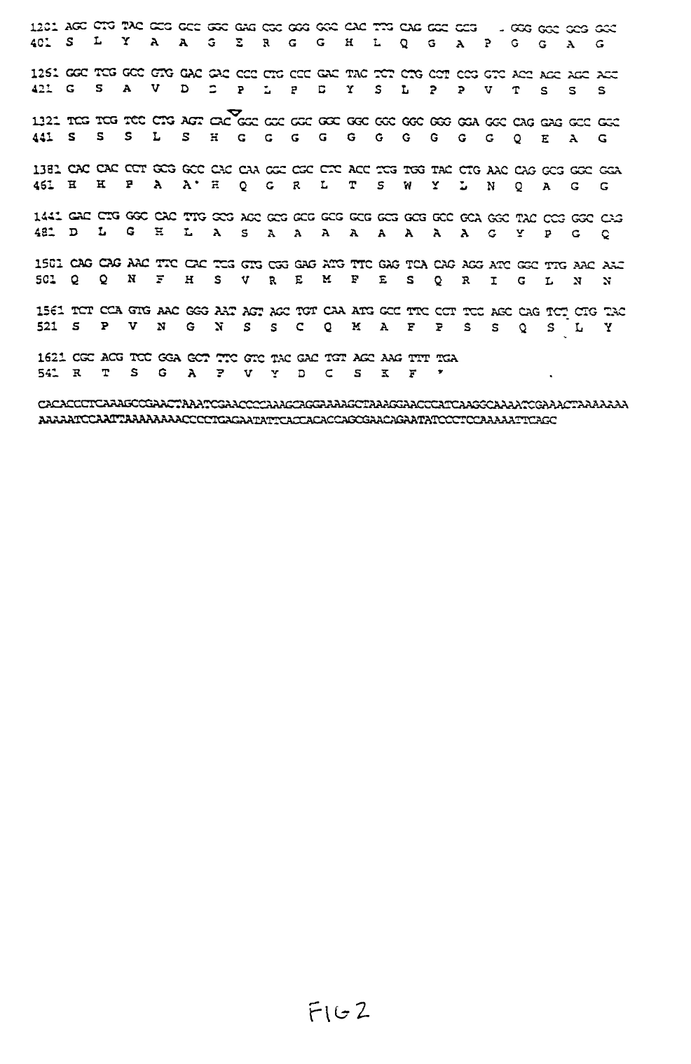

When a crude extract is found to modulate or mimic FREAC3 biological activity, further fractionation of the positive lead extract is necessary to isolate chemical constituents responsible for the observed effect. Thus, the goal of the extraction, fractionation, and purification process is the careful characterization and identification of a chemical entity within the crude extract having an activity that prevents or ameliorates anterior segment disorder or glaucoma, via the modulation or mimicry of FREAC3 biological activity or expression. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may subsequently be analyzed using a standard animal model for anterior segment dysgenesis or glaucoma. One such model is a mouse that has one or both Mf1 (murine FREAC3) genes knocked out or mutated at the positions corresponding to those described herein for FREAC3 (see FIG. 2). Another such model is a mouse (either wild-type, or with knocked out or mutated Mf1 genes) that contains a mutated human FREAC3 transgene.

Screens for Compounds that Modulate FREAC3 mRNA or Protein Expression

FREAC3 cDNAs may be used to facilitate the identification of compounds that increase or decrease FREAC3 protein expression. In one approach, candidate compounds are added, in various concentrations, to the culture medium of cells expressing FREAC3 mRNA. The FREAC3 mRNA expression is then measured, for example, by Northern blot analysis (F. Ausubel et al., Current Protocols in Molecular Biology, John Wiley & Sons, New York, N.Y., 1994) using a FREAC3 DNA, cDNA, or RNA fragment as a hybridization probe. The level of FREAC3 mRNA expression in the presence of the candidate compound is compared to the level of FREAC3 mRNA expression in the absence of the candidate compound, all other factors (e.g., cell type and culture conditions) being equal. Cells that normally express FREAC3, such as those derived from skeletal muscle, heart, liver, kidney, pancreas, prostate, testes, ovary, fetal kidney, and peripheral blood leukocytes may be used. Moreover, cells whose FREAC3 promoter is not normally active may be provided with an exogenously-derived FREAC3 promoter fused to FREAC3 or to a reporter gene, for example, luciferase or β-galactosidase (see below), and used in the assays described herein.

As an alternative approach to detecting compounds that regulate FREAC3 at the level of transcription, candidate compounds may be tested for the ability to regulate the expression of a reporter gene whose expression is directed by a FREAC3 gene promoter. For example, a cell that normally expresses FREAC3, such as a cell derived from skeletal muscle, or alternatively, a cell that normally does not express FREAC3, such as a cell derived from colon, may be transfected with a expression plasmid that includes a luciferase (or other reporter) gene operably linked to the FREAC3 promoter. Candidate compounds may then be added, in various concentrations, to the culture medium of the cells. Luciferase expression levels may then be measured by subjecting the compound-treated transfected cells to standard luciferase assays known in the art (such as the luciferase assay system kit used herein that is commercially available from Promega), and rapidly assessing the level of luciferase activity on a luminometer. The level of luciferase expression in the presence of the candidate compound is compared to the level of luciferase expression in the absence of the candidate compound, all other factors (e.g., cell type and culture conditions) being equal. An increase in luciferase expression indicates a compound that increases FREAC3 gene expression; conversely, a decrease in luciferase expression indicates a compound that decreases FREAC3 gene expression. The effect of candidate compounds on FREAC3-mediated gene expression may, instead, be measured at the level of translation by using the general approach described above with standard protein detection techniques, such as Western blotting or immunoprecipitation with a FREAC3-specific antibody (for example, the FREAC3-specific antibody described herein).

Screens for Compounds that Modulate or Mimic FREAC3 Biological Activity

Compounds may also be screened for their ability to modulate mutant or wild-type FREAC3 biological activity, for example, transcriptional activation of a target gene by FREAC3. In this approach, the level of FREAC3-mediated transcription in the presence of a test compound is compared to the level of transcription in the absence of the test compound, under equivalent experimental conditions. Again, the screen may begin with a pool of candidate compounds, from which one or more useful modulator compounds are isolated in a step-wise fashion. Transcriptional activation of a target gene by FREAC3 may be measured by any standard assay, for example, those described herein.

Another method for detecting compounds that modulate the biological activity of FREAC3 is to screen for compounds that interact physically with a given FREAC3 polypeptide. These compounds are detected by adapting yeast two-hybrid expression systems known in the art. These systems, which detect protein interactions using a transcriptional activation assay, are generally described by Gyuris et al. (Cell 75:791-803, 1993) and Field et al. (Nature 340:245-246, 1989), and are commercially available from Clontech (Palo Alto, Calif.).

Below are examples of high-throughput systems useful for evaluating the efficacy of a molecule or compound in treating or preventing anterior segment dysgenesis and/or glaucoma caused by a mutant FREAC3 protein, or whose course is affected by a wild-type FREAC3 protein.

Reporter Gene Assays for Compounds that Modulate or Mimic FREAC3 Transcriptional Activity

Assays employing the detection of reporter gene products are extremely sensitive and readily amenable to automation, making them ideal for the design of high-throughput screens.

Cloned DNA fragments encoding a transcriptional control region the activity of which is regulated by FREAC3, are easily inserted, by DNA subcloning, into a reporter gene vector, thereby placing a vector-encoded reporter gene under the transcriptional control of the FREAC3-regulated transcriptional contol region. The transcriptional activity of a promoter operatively linked to a reporter gene can then be directly observed and quantitated as a function of reporter gene activity in a reporter gene assay. Such plasmid or viral reporter gene vectors contain cassettes encoding reporter genes such as lacZ/β-galactosidase, green fluorescent protein, and luciferase, among others. Assays for reporter gene activity may employ, e.g., calorimetric, chemiluminescent, or fluorometric detection of reporter gene products.

At appropriate timepoints, cells treated with test compounds are lysed and subjected to the appropriate reporter assays, for example, a colorimetric or chemiluminescent enzymatic assay for lacZ/β-galactosidase activity, or fluorescent detection of green fluorescent protein (GFP). Changes in reporter gene activity of samples treated with test compounds, relative to reporter gene activity of appropriate control samples, indicate the presence of a compound that modulates the transcriptional activity of FREAC3.

In one embodiment, a FREAC3-activated gene construct could include a reporter gene such as lacZ or green fluorescent protein (GFP), operably linked to a promoter from a gene that is transcriptionally activated by FREAC3. Alternatively, an artificial FREAC3-activated gene may be created by fusing multiple copies of an artificial FREAC3 binding site that is known in the art (aGTAAA(T/c)AAAca; (SEQ ID NOs: 3 and 4) upstream from a minimal promoter, such as the herpes simplex thymidine kinase promoter. These regulatory sequences may be fused to a downstream reporter gene (e.g., lacZ), and a test compound-modulated alteration in binding of FREAC3 to the FREAC3 binding site will be observed as a change in the level of reporter gene activity.

A FREAC3-activated gene construct may be present within the genomic DNA of a cell to be used for analyzing a test compound, or may be transiently introduced. A second gene construct, comprising a second reporter gene operably linked to a second promoter (such as an SV40 promoter), is included as an internal control. Hence, the change in reporter gene activity of a reporter gene operably linked to a transcriptional control sequence that is a target of FREAC3 reflects the ability of a test compound to modulate the transcriptional activity of FREAC3.

FREAC3 may be naturally expressed within the test cell, such as a cell derived from skeletal muscle, heart, kidney, pancreas, prostate, testes, ovary, peripheral blood leukocytes, or fetal kidney, or may be artificially expressed from a permanently- or transiently-introduced FREAC3-encoding nucleic acid; nucleic acids encoding either wild-type or mutant forms of FREAC3 may be used. As well, reporter gene assays can be performed in cells lacking FREAC3, in order to isolate molecules that mimic FREAC3 activity. In order to identify compounds that increase or decrease transcription of the FREAC3 gene itself, reporter gene constructs employing the FREAC3 promoter region may be used.

Enzyme-Linked Immunosorbant Assays for Compounds that Modulate or Mimic FREAC3 Transcriptional Activity

Enzyme-linked immunosorbant assays (ELISAs) are easily incorporated into high-throughput screens designed to test large numbers of compounds for their ability to modulate biological activity of a given protein. When used in the methods of the invention, changes in the level of a given indicator protein (e.g., the product of a gene that is transcriptionally activated by FREAC3), relative to a control, reflects a compound that modulates FREAC3 biological activity (or that mimics FREAC3 activity, depending upon the assay). The presence of FREAC3 polypeptide also may be monitored in order to test for compounds that influence FREAC3 transcription, translation, or mRNA or polypeptide stability. The test samples may be cells, cell lysates, or purified or partially-purified molecules. Cells may be derived from heart, skeletal muscle, kidney, pancreas, prostate, testis, ovary, fetal kidney, or peripheral blood leukocytes, or may be other types of cells that are genetically engineered to express FREAC3 via a permanently- or transiently-introduced FREAC3-encoding gene.

Protocols for ELISA may be found, for example, in Ausubel et al., Current Protocols in Molecular Biology, John Wiley & Sons, New York, N.Y., 1998. In one embodiment, the so-called “sandwich” ELISA, treated samples comprising cell lysates or purified molecules are loaded onto the wells of microtiter plates coated with “capture” antibodies. Unbound antigen is washed out, and a second antibody, coupled to an agent to allow for detection, is added. Agents allowing detection include alkaline phosphatase (which can be detected following addition of calorimetric substrates such as p-nitrophenolphosphate), horseradish peroxidase (which can be detected by chemiluminescent substrates such as ECL, commercially available from Amersham) or fluorescent compounds, such as FITC (which can be detected by fluorescence polarization or time-resolved fluorescence).

The amount of antibody binding, and hence the level of indicator protein expressed by a gene that is transcriptionally activated by FREAC3, is easily quantitated on a microtiter plate reader. For example, an increased level of an indicator protein in a treated sample, relative to the level of an indicator protein in an untreated sample, indicates a test compound that increases the transcriptional activity of FREAC3. It is understood that appropriate controls for each assay are always included as a baseline reference.

High-throughput assays for the purpose of identifying compounds that modulate or mimic FREAC3 biological activity can be performed using treated samples of cells, cell lysates, baculovirus lysates, and purified or partially-purified molecules.

Interaction Trap Assays

Two-hybrid and one-hybrid methods, and modifications thereof, are used to screen for compounds that modulate the physical interactions of FREAC3 with other molecules (e.g., proteins or nucleic acids). Such assays may also be used to identify novel proteins that interact with FREAC3, and hence may be naturally occurring regulators of FREAC3. Such assays are well-known to skilled artisans, and may be found, for example, in F. Ausubel et al., Current Protocols in Molecular Biology, John Wiley & Sons, New York, N.Y., 1998.

DNA Binding Assays

Binding of mutant or wild-type FREAC3 to the FREAC3 in vitro binding site sequence (aGTAAA(T/c)AAAca; SEQ ID NOs: 3 and 4) may be used to screen for compounds that modulate FREAC3 biological activity. One method by which to quantitate such changes is by an ELISA-type assay. Samples containing FREAC3 are incubated with test compounds as described above, plus an oligonucleotide encoding a FREAC3 binding site (such as aGTAAA(T/c)AAAca) that is affixed to a solid support (e.g., a filter, or a microtiter well). After allowing FREAC3 to interact with its cognate binding sequence and washing away unbound FREAC3, the amount of FREAC3 bound to the immobilized oligonucleotide may be quantitated by subsequent incubation with a labeled antibody. A compound that increases or decreases the amount of mutant FREAC3 bound to the immobilized oligonucleotide indicates a compound that may be useful for the treatment or prevention of glaucoma or anterior segment dysgenesis.

Secondary Screens of Test Compounds that Appear to Modulate or Mimic FREAC3 Transcriptional Activity

After test compounds that appear to have FREAC3-modulating activity are identified, it may be necessary or desirable to subject these compounds to further testing. The invention provides such secondary confirmatory assays. For example, a compound that appears to modulate the biological activity of mutant FREAC3 (i.e., induces mutant FREAC3 to have activity approaching wild-type FREAC3) in early testing may be subject to additional assays to determine the effect of the compound on wild-type FREAC3.

At late stages testing is performed in vivo to confirm that compounds initially identified as affecting FREAC3 activity have the predicted effect on FREAC3 in vivo. In the first round of in vivo testing, the compound is administered to animals with either wild-type or mutant FREAC3 genes by one of the means described in the Therapy section below. Eye tissue, or other tissues that express FREAC3 (i.e, see FIG. 2) is isolated within hours to days following treatment, and are subjected to assays as described above.

Construction of Transgenic Animals and Knockout Animals

FREAC3 knockout animals, such as FREAC3 knockout mice, may be developed by homologous recombination. Animals that overproduce mutant FREAC3 may be generated by integrating one or more FREAC3 sequences into the genome of such animals, according to standard transgenic techniques.

A replacement-type targeting vector, which could be used to create a knockout model, may be constructed using an isogenic genomic clone, for example, from a mouse strain such as 129/Sv (Stratagene Inc., LaJolla, Calif.). The targeting vector may be introduced into embryonic stem (ES) cells by electroporation to generate ES cell lines that carry a profoundly truncated form of a FREAC3 gene. To generate chimeric founder mice, the targeted cell lines are injected into a mouse blastula-stage embryo, and mice that transmit the FREAC3 knockout gene to their offspring are identified. Heterozygous FREAC3 knockout mice may be bred to homozygosity, such that no FREAC3 is expressed. Knockout mice provide the means, in vivo, to screen for therapeutic compounds that modulate anterior segment dysgenesis or the onset or progression of glaucoma via a FREAC3-dependent or FREAC3-affected pathway.

Therapeutic Use of Compounds Identified by High Throughput Systems

A compound that promotes an alteration in the expression or biological activity of the FREAC3 protein is considered particularly useful in the invention; such a molecule may be used, for example, as a therapeutic to increase cellular levels of biologically active FREAC3 and thereby exploit the role of FREAC3 polypeptides in anterior segment formation, differentiation of the trabecular meshwork, and intraocular pressure regulation. This would be advantageous in the prevention and/or treatment of anterior segment dysgenesis and/or glaucoma.

Molecules that are found, by the methods described above, to effectively modulate FREAC3 gene expression or polypeptide activity may be tested further in animal models described above. If they continue to function successfully in an in vivo setting, they may be used as therapeutics to enhance FREAC3 biological activity and/or expression, as appropriate.

Compounds identified using any of the methods disclosed herein, may be administered to patients or experimental animals with a pharmaceutically-acceptable diluent, carrier, or excipient, in unit dosage form, as described in the Therapy section below.

Therapy

Therapeutic molecules identified using any of the methods disclosed herein may be administered to patients or experimental animals with a pharmaceutically-acceptable diluent, carrier, or excipient, in unit dosage form. Conventional pharmaceutical practice may be employed to provide suitable formulations or compositions to administer such compositions to patients or experimental animals. Although intravenous administration is preferred, any appropriate route of administration may be employed, for example, parenteral, subcutaneous, intramuscular, intracranial, intraorbital, ophthalmic, intraventricular, intracapsular, intraspinal, intracisternal, intraperitoneal, intranasal, aerosol, or oral administration. Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found in, for example, “Remington's Pharmaceutical Sciences.” Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for administering molecules of the invention include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The following examples are to illustrate the invention. They are not meant to limit the invention in any way.

### EXAMPLE 1

### General Methods

Clinical Data

The clinical presentations within IRID1 Families 1-5 have all been reported previously. Families 1 and 2 were originally diagnosed with iridogoniodysgenesis anomaly (IGDA) (Mears et al., Am. J. Hum. Genet. 59:1321-7, 1996; Pearce et al., Can. J. Ophthalmol. 18:7-10, 1983), Family 3 with Axenfeld-Rieger Anomaly (Gould et al., Am. J. Hum. Genet. 61:765-768, 1997), Family 4 with familial iridogoniodysplasia (Jordan et al., Am J Hum Genet 61, 1997) and Family 5 with goniodysgenesis and glaucoma (Morissette et al., Am J Hum Genet 61:A286, 1997). All five IRID1 families demonstrate phenotypic variability but affected individuals typically present with iris hypoplasia, iridocorneal angle defects (goniodysgenesis), and increased intraocular pressure with subsequent risk of glaucoma. Affected individuals within Family 3 additionally presented with a prominent, anteriorly displaced Schwalbe's line (posterior embryotoxon) attached to peripheral iris strands bridging the iridocorneal angle, and displaced pupils (corectopia). Some of the patient families also have a history of congenital heart defects. Sixteen unrelated individuals presenting with anterior segment dysgenesis were also studied. The study and collection of blood samples from all individuals included in this report were approved by the Research Ethics Board of the Faculty of Medicine of the University of Alberta.

Polymorphic Markers

Novel polymorphisms were detected in exon 5 of the NAD(P)H:quinone oxidoreductase-2 gene (NQO2) by direct sequencing of PCR products amplified from key recombinant branches of the IRID1 families. Primers for exon 5: forward 5′-gcttcattccgaatcaccag-3′ (SEQ ID NO: 5), reverse 5′-gtcccctccctccaactatc-3′ (SEQ ID NO: 6). Primers were designed using Primer3, available from the Whitehead Institute for Biomedical Research (http://www-genome.wi.mit.edu/cgi-bin/primer/primer3.cgi). The two polymorphisms within NQO2 exon 5 both affect MspI sites at positions 111 bp and 188 bp of the 250 bp PCR product, generating a four-allele polymorphic system.

Physical Mapping

The preliminary physical map for the 6p25 IRID1 region was obtained from the Whitehead Institute for Biomedical Research web site (http://www-genome.wi.mit.edu/). The human bacterial artificial chromosome (BAC) library (Kim et al. Genomics 34:213-218, 1996; Shizuya et al. Proc. Natl. Acad. Sci. USA 89:8794-8797, 1992) was screened by PCR with STSs/ESTs mapped to the region, according to Research Genetics protocols. Selected clones were mapped by fluorescence in situ hybridization (FISH) to confirm cytogenetic location, and then analysed for STS content to determine order and overlap between clones.

Sequence Scanning

Sequence scanning was performed on the BAC RMC06B016. This BAC, containing an insert of approximately 150 kb in size, was sheared randomly and fragments ranging from 2-3 kb were subcloned into M13 mp 18 vector. Sequences were obtained from 509 subclones using ABI 373 and 377 automated sequencers and were assembled into contigs using Seqman (DNASTAR). Contigs were searched for coding sequence using BLAST 2.0 against the GenBank and dEST databases. GRAIL 1.2 was used to predict coding sequence not represented in existing databases.

Mutation Detection

Fragments were amplified from the single-exon FREAC3 gene, using primers designed by Primer3 (See Table 1). Dimethyl sulfoxide (final concentration of 5-10%) was added to PCR reactions to alleviate secondary-structure problems created by the very high GC-content of FREAC3. PCR products were purified with QIAquick columns (QIAGEN, Los Angeles, Calif.) then directly sequenced via 33-P cycle sequencing (Amersham, Malvern, Pa.).

Mutations were confirmed in affected individuals and screened for in a 100 control chromosomes by the following methods: the 10 bp deletion (del nt 91-100) was detected through analysis of PCR products on 1.5% agarose/1.5% NuSieve electrophoretic gels. The G245C mutation was detected through loss of an Alu I site. The C261G mutation was detected through generation of a Bsp HI site.

The insertion polymorphisms (GGC375ins and GGC347ins) were detected by sequencing in both patients and control individuals.

Expression Analysis

Expression of FREAC3 was determined by Northern blot analysis of commercially available filters (Clontech, Palo Alto, Calif.) that contained poly(A)+-selected RNA from a variety of adult and fetal tissues. To avoid cross-hybridization with other forkhead-related genes, the probe for FREAC3 was selected from the 3′ region (nucleotides 1192-1690; see FIG. 2). Hybridization and washes were performed according to the manufacturer's protocols. The human β-actin control probe, provided by the manufacturers, was used to equalize loading differences.

Mf1 is the mouse homologue of the FREAC3 gene. A Mf1 knockout mouse was generated by homologous recombination in embryonic stem cells in which sequences corresponding to amino acids 50-553 and the 3′ untranslated region of the Mf1 gene were replaced by a lacZ/PGKneor cassette in frame with the first AUG. Expression of the Mf1LacZ gene was detected by X-Gal staining of eye sections from Mf1LacZ/+(+/−) and Mf1LacZ/Mf1LacZ (−/−) mouse embryos (14.5 dpc).

### EXAMPLE 2

### Genetic Refinement of the Location of the IRID1 Locus

Genetic linkage analysis was used to refine the location of IRID1 locus. FIG. 1 shows a schematic diagram of chromosome 6, illustrating the genetic mapping of the IRID1 gene(s). Cumulative genetic distances (in cM) from the telomere are indicated to the left. The disease haplotypes cosegregating in the five IRID1 families are represented by the filled rectangles. Key individuals are identified at the top of the figure, with disease status indicated at the bottom. The location of the locus associated with anterior segment dysgenesis and glaucoma in families 1 and 4 is indicated to the right. Results from analyses of known and novel 6p25 polymorphic markers in five IRID1 families were generally consistent with the localization of IRID1 between D6S1600 and polymorphisms in the NAD(P)H:quinone oxidoreductase (NQO2) gene (FIG. 1). However, one unaffected individual (IRID1 family 1; VIII:1) had an apparent crossover event placing IRID1 distal to D6S344 (FIG. 1). This observation is inconsistent with FREAC3 being a candidate gene for IRID1 in this family (see below). IRID1 is thought to be a fully penetrant autosomal dominant disorder. Nevertheless, non-penetrance of IRID1 in individual VIII:1 could not formally be ruled out as a possible explanation of this apparent mapping discrepancy.

### EXAMPLE 3

### FREAC3, a Candidate Gene Located in the IRID1 Critical Region

In order to physically clone the IRID1 interval, twenty-nine BACs were obtained by screening a BAC genomic library with known sequence-tagged sites (STSs) and expressed sequence tags (ESTs). BAC RMC06B016 was found to contain the distal flanking marker D6S344 and to test positive with primers designed from published partial sequence of FREAC3, a gene previously mapped to 6p25 (Larsson et al., Genomics 30: 464-469, 1995). FREAC3 is a member of the forkhead transcription factor gene family shown to be involved in development, cell-specific development, and oncogenesis. FREAC3 was reported as located within 20 kb of the 6p25 translocation breakpoint in an individual with an unbalanced (t(2,6) (q35, p25)) karyotype who presented with a variety of clinical findings including glaucoma (Nishimura et al., Am. J. Hum. Genet. 61:A21, 1997).

The DNA sequence of approximately 80% of BAC RMC06B016, or about 120 kb of sequence, was determined as a rapid means of characterizing FREAC3 and of identifying additional genes within the IRID1 critical region. The BAC RMC06B016 forkhead-like region was identical to the partial DNA sequence of the FREAC3 gene. Additional sequence analysis revealed that FREAC3 has an intronless open reading frame of 1659 bp (SEQ ID NO: 1) and is predicted to encode a protein (SEQ ID NO: 2) of 553 amino acids (FIG. 2).

Mf1, the murine gene homologous to FREAC3, is also predicted to encode a protein 553 amino acids in length and has been mapped to mouse chromosome 13 in a region of conserved synteny with human 6p25 (Database, M. G. Mouse Genome Informatics, The Jackson Laboratory, Bar Harbor, Me., 1998, URL: http://www.informatics.jax.org/). The human FREAC3 gene and the mouse Mf1 gene share 89% of their nucleotide sequence through the coding region with the highest degree of identity (96%) seen over the 330 nucleotides of the forkhead domains. Overall, identity at the protein level was found to be 92% with 100% identity throughout the forkhead DNA-binding region.

### EXAMPLE 4

### FREAC3 Mutations in Patients with Anterior Segment Dysgenesis and Glaucoma

The FREAC3 gene was screened for mutations by direct DNA sequencing of PCR products from affected individuals of the five IRID1 families linked to 6p25 polymorphic loci and in 16 additional unrelated individuals with anterior segment dysgenesis. Five nucleotide alterations of FREAC3 were found. FIG. 2 shows the nucleotide and predicted amino acid sequence of FREAC3. The open-reading frame is 1659 bp in length, predicted to encode a 553 amino acid protein. The forkhead domain, spanning amino acids 69-178, is boxed. The arrowheads indicate the two locations of the polymorphic GGC insertions (see below).

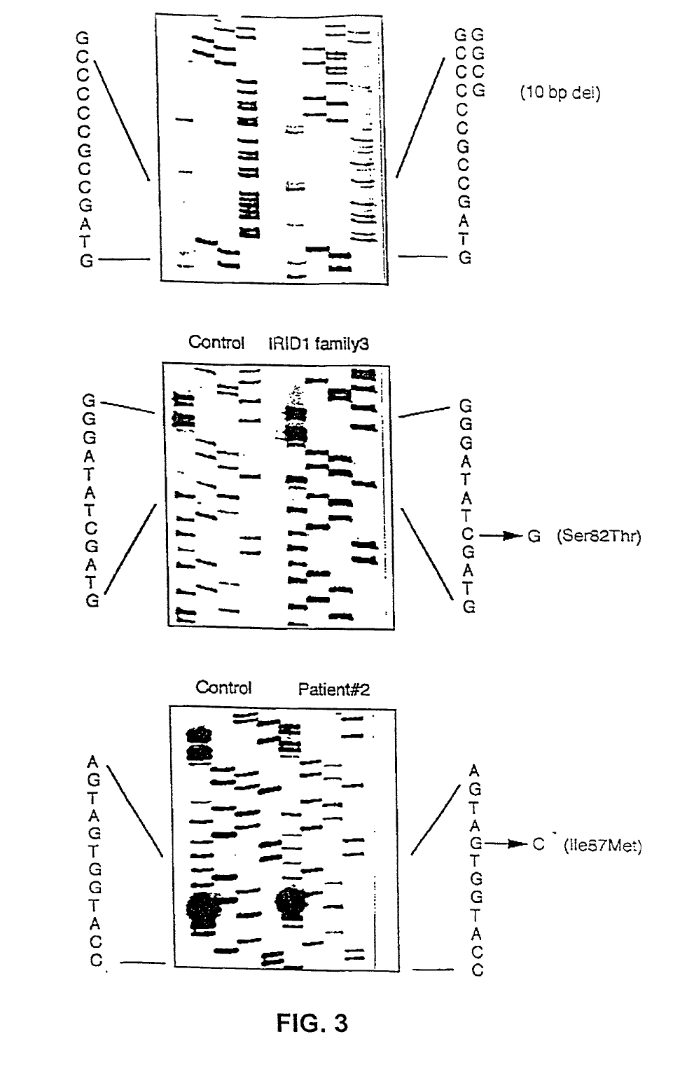

Three FREAC3 mutations detected in patients with anterior segment dysgenesis and glaucoma are indicated by 1, 2, and 3 (FIG. 2); horizontal bars above the nucleotide sequence indicate the affected nucleotides. A 10 base pair deletion of bp 93-102, which is 5′ of the region encoding the FREAC3 forkhead domain, was found in an individual with Axenfeld-Rieger Anomaly (ARA) and glaucoma (Patient #1; FIG. 3). This alteration occurs after the initiation codon, and is predicted to result in a frameshift mutation and premature stop after 10 amino acids. A second alteration, a G to C transversion at nucleotide position 245 resulting in a Ser82Thr mutation in helix 1 of the FREAC3 forkhead domain, was identified in IRID1 family 3 (originally diagnosed with ARA; FIG. 3). This G245C mutation abolished an Alu I restriction enzyme site and was observed to segregate with the anterior segment dysgenesis/glaucoma phenotype in all affected members in Family 3. This amino acid position is invariantly a serine in more than 80 forkhead-family genes from yeast to humans. The distantly-related QRF1 (glutamine Q-rich factor 1) gene has a threonine instead of a serine residue at this position within helix 1. However, as the QRF1 DNA-binding domain is only 84 amino acids in length as compared to 110 amino acids for forkhead genes, QRF1 could well fall outside of the forkhead gene family. Consistent with this notion, QRF1 appears to bind DNA differently from that predicted for forkhead proteins, and therefore may not require a serine at this position, unlike all other forkhead genes. Site-directed mutagenesis of this serine and the two flanking tryrosines in the related forkhead gene HNF-3γ abolished DNA-binding activity. The third mutation, a C to G transversion at nucleotide position 261 that would result in the missense mutation Ile87Met in helix 1 of the FREAC3 forkhead domain (FIG. 2), was identified in an individual diagnosed with ARA and glaucoma (Patient #2; FIG. 3). This C261G mutation creates a Bsp HI restriction enzyme site. This position within helix 1 is an isolucine in over 88% of forkhead genes, and has never been reported as a methionine. Interestingly, as well as occurring within the putative DNA-binding domain of FREAC3, both the Ser82Thr and Ile87Met missense mutations occur within a conserved region shown to act as a nuclear localization signal necessary and sufficient for nuclear targeting of the related forkhead family gene, HNF-3β. These three FREAC3 nucleotide alterations were not observed in over 100 unaffected chromosomes from normal controls.

FIG. 3 shows autoradiographs of sequence analysis of the mutations identified in IRID1 Family 3 and in two patients with anterior segment dysgenesis and glaucoma. PCR products were amplified from patient DNA samples and directly sequenced. Normal sequences are shown to the left, sequences from affected individuals are shown to the right. The reverse primer sequence is shown in each case with the lanes representing bases GATC from left to right. Positions of the mutations are shown to the right and predicted effects of FREAC3 DNA mutations are indicated to the far right.

Two alterations, GGC375ins and GGC447ins, each involving the insertion of an extra GGC triplet in two separate GGC repeats within the FREAC3 coding region (FIG. 2) were found in both patients and control individuals. These alterations are therefore presumed to be non-IRID1-associated polymorphisms of FREAC3.

### EXAMPLE 5

### FREAC3 Expression Studies

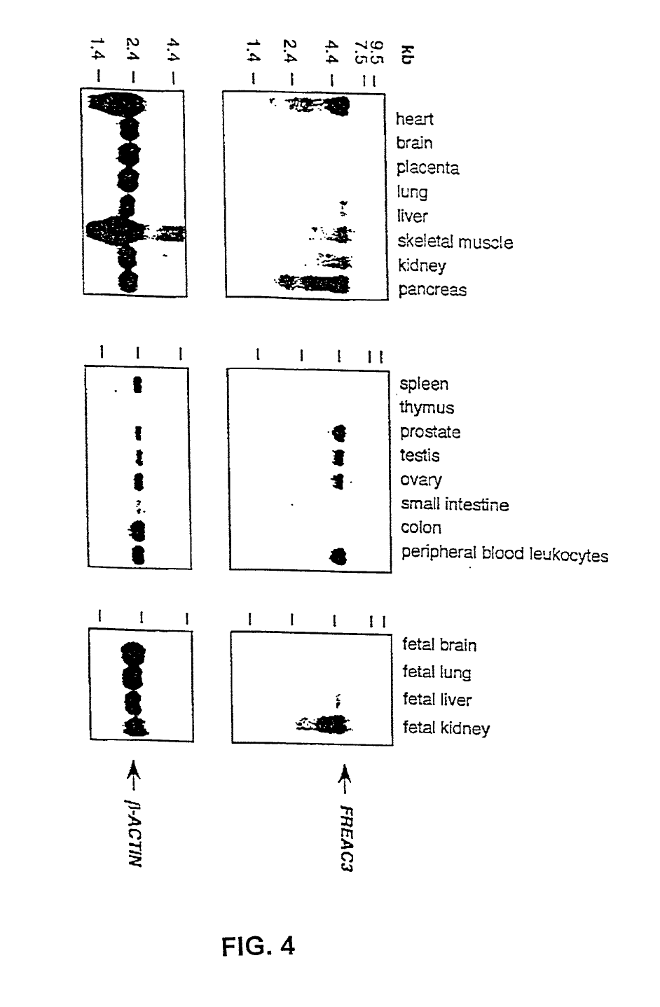

FIG. 4 shows a Northern blot analysis for FREAC3 expression in human, adult, and fetal tissues. Filters were hybridized with a FREAC3 probe (upper panels), and a β-actin control probe (lower panels). A 4.4 kb FREAC3 mRNA transcript was detected by Northern blot analysis and found to be widely expressed in adult and fetal human tissues. Highest expression of FREAC3 was observed in adult kidney, heart and peripheral blood leukocytes, and in fetal kidney (FIG. 4). An alternative transcript of size 4.0 kb was also detected in fetal kidney, possibly suggesting an alternative promoter or polyadenylation site being used in this tissue. PCR analyses indicate that FREAC3 is also expressed in human fetal cranial facial RNA and in the adult iris.

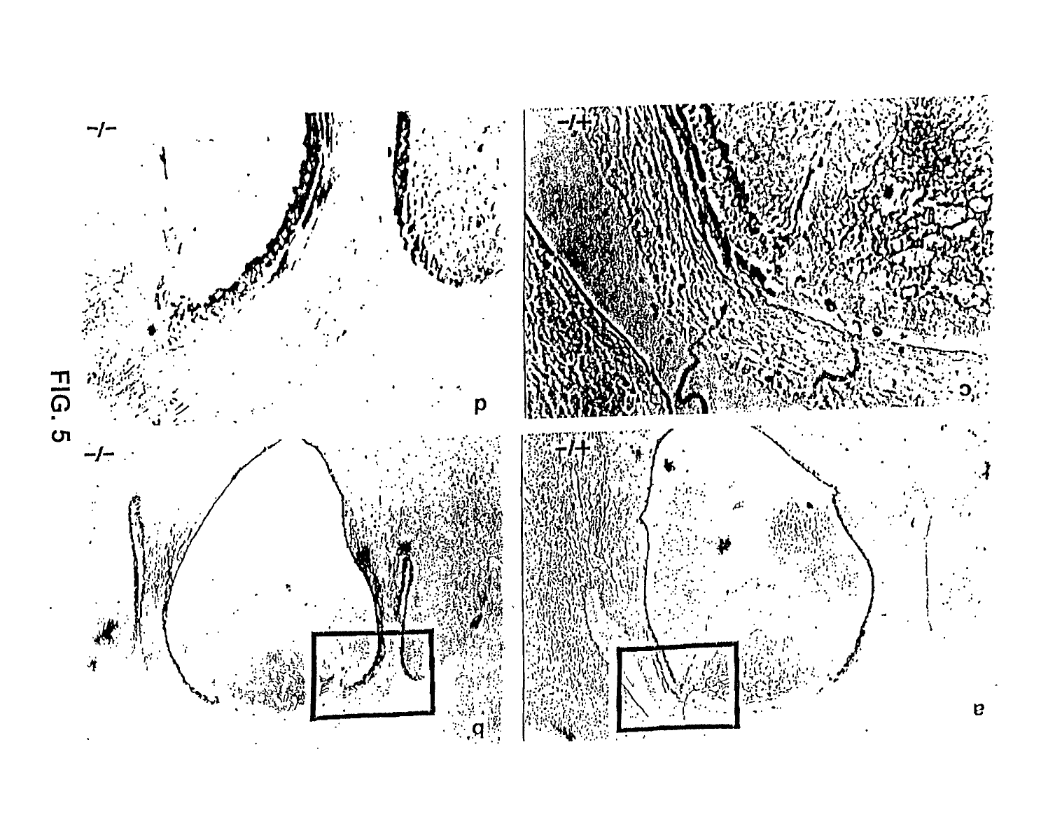

FIGS. 5(a-d) shows the expression pattern of Mf1LacZ in Mf1LacZ homozygous and heterozygous embryos. Mf1 expression in Mf1LacZ/+(+/−) and Mf1LacZ/Mf1LacZ (−/−) embryos is indicated by the lacZ staining observed in photographs of sections of the developing eye in 14.5 dpc mice. The boxed regions of panels 5a and 5b, respectively, are shown magnified in panels 5c and 5d. Blue-stained tissue indicates regions of lacZ expression, which correspond to abundant Mf1 expression in the periocular mesenchyme, developing lids and anterior segment.

In the developing eye, lacZ staining was abundant in the periocular mesenchyme, in the developing lids and anterior segment (FIGS. 5(a-d)). LacZ activity was also observed in the mesenchyme of the hindlimb, heart, and in the perichondrium of the ribs. The expression pattern of the murine homologue of the FREAC3 gene and the fact that Mf1 homozygous knockout mice develop severe eye anomalies and hydrocephalus, are strongly consistent with the hypothesis that FREAC3 has a role in eye development. The relatively less severe anomalies observed in human IRID1 patients as compared to the Mf1 homozygous knockout mice presumably result from the fact that IRID1 patients are heterozygotes and thus retain a single functional copy of the FREAC3 gene.

### EXAMPLE 6

### IRID1 is Genetically Heterogeneous

Complete DNA sequencing of the FREAC3 gene coding region in affected individuals of Families 1, 2, 4, and 5 surprisingly failed to identify any IRID1-associated mutations of FREAC3. In addition, analysis of the GGC347ins polymorphism in IRID1 Family 4 genetically excluded the FREAC3 gene from underlying IRID1 in this family (FIG. 1). The recombination event in VIII:24 of IRID1 Family 4 together with the recombination event within the unaffected individual VIII:1 in IRID1 Family 1 discussed previously are consistent with the localization of the second IRID1 locus between D6S1600 and D6S344 (FIG. 1).

Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth, and follows in the scope of the appended claims.

What is claimed is:

1. A method of diagnosing a mammal for an increased likelihood of developing a disease of the eye, said method comprising analyzing nucleic acid of said mammal to determine whether said nucleic acid contains a mutation in a FREAC3 gene, wherein the presence of said mutation is an indication that said mammal has an increased likelihood of developing a disease of the eye.

2. The method of claim 1, wherein said mutation is a missense mutation.

3. The method of claim 2, wherein said mutation results in a truncated protein.

4. The method of claim 1, wherein primers are used for detecting said mutation.

5. The method of claim 1, wherein said analyzing includes detecting the loss of a recognition site for a restriction endonuclease.

6. A kit for the analysis of FREAC3 nucleic acid, said kit comprising nucleic acid probes for analyzing the nucleic acid of a mammal, wherein said analyzing is sufficient to determine whether the mammal contains a mutation in said FREAC3 nucleic acid.

7. A method of diagnosing a mammal for an increased likelihood of developing a disease of the eye, said method comprising detecting the presence of a mutant FREAC3 polypeptide in said mammal, wherein the presence of said mutant FREAC3 polypeptide indicates that said mammal has a mutation in a FREAC3 gene, wherein the presence of said mutation is an indication that said mammal has an increased likelihood of developing a disease of the eye.

8. The method of claim 1, wherein said mammal is a human.

9. Nucleic acid encoding mutant FREAC3, wherein said nucleic acid has at least one mutation, wherein said mutation is an indication that a mammal from which said nucleic acid is derived has an increased likelihood of developing glaucoma.

10. A method of detecting a compound useful for the prevention or treatment of a disease of the eye, said method comprising assaying transcription levels of a reporter gene operably linked to a promoter, said promoter comprising a FREAC3 binding site, said method comprising the steps of:
(a) exposing said reporter gene to said compound, and
(b) assaying said reporter gene for an alteration in reporter gene activity relative to a reporter gene not exposed to said compound.

11. The method of claim 1, wherein said disease of the eye is glaucoma.

12. The method of claim 7, wherein said disease of the eye is glaucoma.

13. The method of claim 10, wherein said disease of the eye is glaucoma.

14. A method of treating a disease of the eye by in vivo gene therapy, said method comprising introducing into the cells of the eye a nucleic acid that encodes wild-type FREAC3, wherein said nucleic acid is operably linked to regulatory sequences for expression of said FREAC3, wherein said regulatory sequences comprise a promoter, and wherein said expression of said FREAC3 is sufficient to ameliorate symptoms of said disease.
